(19) United States
(12) Patent Application Publication
(10) Pub. No.: US 2007/0038135 A1
(43) Pub. Date: Feb. 15, 2007

(54) Portable physiological parameter detection/display assembly

(75) Inventors: Kang-Ping Lin, Chungli City (TW); Bor-Iuan Jan, Pingdong City (TW); Hao-Yu Jan, Chungli City (TW); Geng-Hong Lin, Pingdong City (TW)

Correspondence Address: Thomas E. Sisson;Jackson Walker, LLP, Suite 2100, 112 E. Pecan, San Antonio, TX, 78205, US

(73) Assignee: Dailycare Biomedical Inc.

(21) Appl. No.: 11/438,410

(22) Filed: May 22, 2006

(30) Foreign Application Priority Data
Aug. 12, 2005 (TW) 094213932

(51) Int. Cl. A61B 5/02

(52) U.S. Cl. 600/500

(57) ABSTRACT

A portable bio parameter detection/display assembly includes an optical sensor for engagement with an operator to acquire a pulse signal, an A/D converter converting the pulse signal into a digital format, a microprocessor for receiving the digital format pulse signal and proceeding with calculation of a pulse rate and at least one heart rate variability parameter, a display to display and a power module to provide necessary electricity to the assembly.

### BACKGROUND OF THE INVENTION

1. Field of the Invention

The present invention relates to a portable physiological parameter detection/display assembly, and more particularly to the portable assembly having the ability to detect the pulse signal based on photoplethysmographic technique in response to the pulse rate and to analyze heart rate variability so as to acquire physiological parameters for reference by the operator.

2. Description of the Prior Art

As the modem society develops, the pace of our daily life goes faster and faster. People have less time to look after their own health due to the pressure coming from the workplace. Especially, repeated delicacies and lack of exercise become the major reasons for diseases such as high cholesterol, hypertension and cardiovascular system malfunction. In addition, diligence in work performance is something to be recommended, however, excessive workload often causes workers “karoshi” or “sudden death”.

Current study shows there is a close relationship between the sudden death and excessive overtime as well as pressure in the workplace, which is because the nerve system of the worker is maintained intense for a long period of time and the mental status is always under high pressure while the worker is at work. Further study indicates that heart rate variability (HRV) may be an index for the valuation of sudden death. According to the study by Huikuri H V et. al. in 1992 and by Sasaki T. et. al. in 1999, the reduce of the heart rate variability is highly related to the increase of having a possibility of sudden death. Because the heart rate variability reflects the heart activities and whether there is an autonomic nervous system problem, when the heart activity is normal and there is no dysautonomia problem, rhythm of the heart may be automatically adjusted by the heart itself in accordance with the physical conditions, which leads to a result that the heart rate variability is high. When the heart activity is unusual and the autonomic nervous system cannot reflect the physical conditions instantly, the heart rate variability is low. Accordingly, the heart rate variability may be used as an index of sudden death.

In order to detect the small fluctuation of the heart rate variability, it is necessary to monitor the physiological parameters. To overcome the shortcomings of the current vital signs detection devices of not having to provide sufficient physiological parameters constantly, the present invention tends to provide an improved portable physiological parameter detection/display assembly to mitigate the aforementioned problems.

### SUMMARY OF THE INVENTION

The primary objective of the present invention is to provide an improved portable physiological parameter detection/display assembly to instantly and constantly provide the required physiological parameters for reference by the operator.

In order to accomplish the aforementioned objective, the portable physiological parameter detection/display assembly of the present invention includes an optical sensor to detect a pulse signal, an A/D converter to convert an analog signal of the pulse signal to digital format, a microprocessor to receive the digital signal and proceed with calculations of parameters of pulse rate and heart rate variability, a display to show the acquired parameters and a power module to provide electricity to the assembly of the present invention.

Other objects, advantages and novel features of the invention will become more apparent from the following detailed description when taken in conjunction with the accompanying drawings.

### BRIEF DESCRIPTION OF THE DRAWINGS

FIG. 1 is a block diagram of the present invention;

FIG. 2 is a schematic plan view showing how the optical sensor works to acquire the pulse signal; and

FIG. 3 is a schematic view showing the application of the optical sensor in a pen.

### DETAILED DESCRIPTION OF THE PREFERRED EMBODIMENT

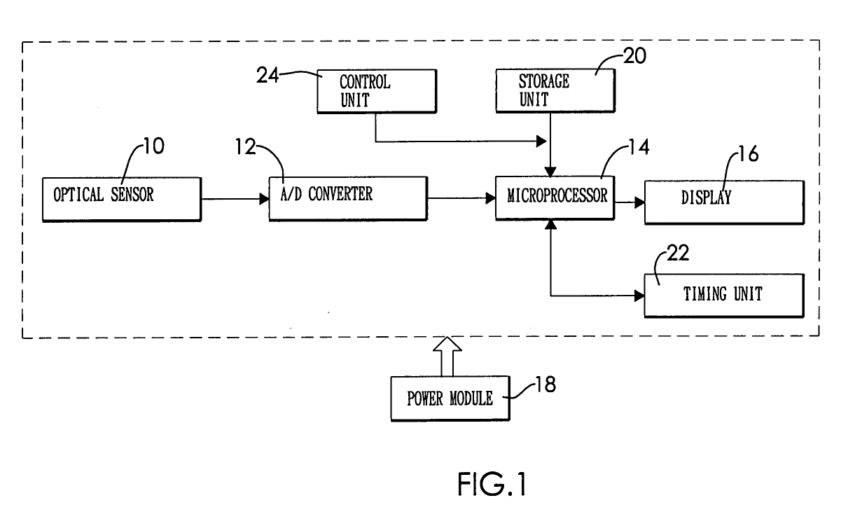

With reference to FIG. 1, it is noted that the portable physiological parameter detection/display assembly in accordance with the present invention includes an optical sensor (10) for engagement with a skin of an operator to acquire the operator's pulse signal, an A/D converter (12) electrically connected to the optical sensor (10) to convert the acquired analog pulse signal to a digital format; a microprocessor (14) electrically connected to the A/D converter (12) to receive the digital signal and proceed with calculation to acquire at least one heart rate variability (HRV) parameter, wherein the HRV analysis is divided into a time-domain analysis and a frequency-domain analysis so as to have multiple choices in the parameter calculation and a display (16) to display the acquired parameters for reference by the operator. Furthermore, a power module (18) is provided to the assembly of the present invention to provide the necessary electricity.

HRV analysis mainly analyzes the variability of heart rate. Recent study shows the HRV level is related to nerve modulation mechanism. Therefore, it is proper to use the HRV as an index for evaluation of physical conditions. According to the standard of heart rate variability signal measurement and analysis set forth by Task Force of the European Society of Cardiology the North American Society of Pacing Electrophysiology in 1996 (Circulation. 1996; 93:1043-1065), the time-domain analysis employs the statistical or geometric methods to analyze the HRV. The parameter acquired from the time-domain analysis includes, for example, Heart rate, mean of NN (normal-to-normal) interval (MEAN), standard deviation (SD) of NN interval, coefficient of variation (CV) of NN interval, the square root of the mean squared differences of successive NN intervals (RMSSD), standard deviation of SD of normal RR intervals (SDSD), HRV triangular index, the triangular interpolation of NN interval histogram (TINN). The frequency-domain analysis is transforming the small fluctuation of heart rate during the observation timeframe into a spectrum so as to enhance the smallest variation. The parameters acquired by the frequency-domain analysis includes high frequency (HF), low frequency (LF), ratio of the HF or the LF over the total power (TP), high frequency power (HFP) and low frequency power (LFP) . . . etc.

With reference to FIG. 1, the portable physiological parameter detection/display assembly of the present invention further includes a storage unit (20), a timing unit (22) and a control unit (24) respectively connected to the microprocessor (14) such that the storage unit (20) is able to store the parameters of HRV acquired from the microprocessor (14), the timing unit (22) is able to provide instant time information during observation period for reference by the operator and the control unit (24) allows the operator to control the control the functions of the entire assembly. For example, the operator is able to select activation, shut-off the signal acquiring process and recall or deleting information in the storage unit (20).

The storage unit (20) may be selected from ROM, RAM, flash memory or an EEPROM. The control unit (24) may be activated via keys, switches or touch-panels. In addition, the power module (18) may be using a solar powered battery or a rechargeable battery.

The reason for using the optical sensor is that when the blood flows through a vessel, the oxygen volume contained in the blood varies in accordance with the heartbeat, which affects the reflection of blood to light. Therefore, the optical sensor is able to acquire a pulse signal, and the pulse signal can reflect the systole and diastole of heart.

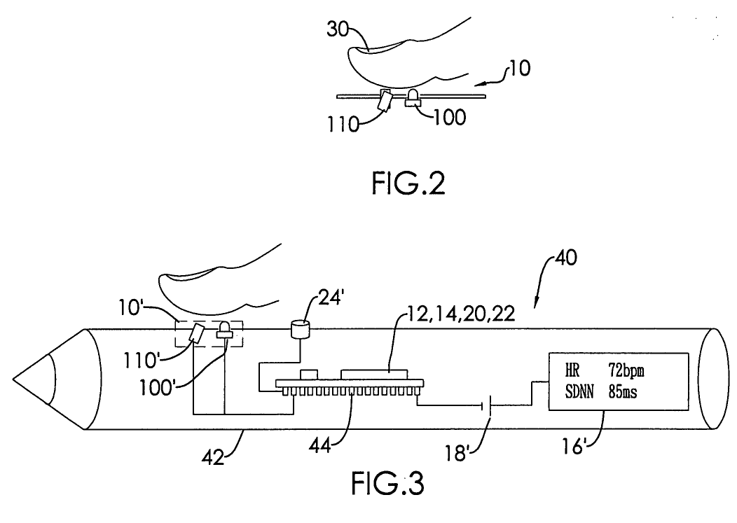

With reference to FIG. 2, it is noted that the optical sensor (10) of the present invention includes at least a light emitter (100) and at least one light detector (110). The light emitter (100) is to generate a light beam projected to the operator's tissue such as ear, hand, chest, finger, etc. In the depiction of the accompany drawing of FIG. 2, the fingertip is used in this embodiment, but is not limited only to this body portion. Therefore, when the light detector (110) detects the reflected light from the tissue, which indicates the oxygen volume in the blood in an entire heartbeat cycle, a pulse signal is then picked up and transmitted to the microprocessor (14) to calculate pulse rate and HRV parameters. Furthermore, the light emitted from the light emitter (100) is red light or infrared light.

With reference to FIG. 3, it is noted that the assembly of the present invention is incorporated with a pen so that a multi-functional pen (40) is generated. The pen (40) includes a tube (42) and the light emitter (100′) and the light detector (110′) of the optical sensor (10′) are mounted on an inner periphery of the tube (42) to enable the operator to easily employ the pen (40) to engage with the operator's skin. The pen (40) further has a circuit board (44) and the A/D converter (12), the microprocessor (14), the storage unit (20) and the timing unit (22), as shown in FIG. 1, may all be mounted on the circuit board (44). Consequently, the pulse rate and HRV parameters calculated by the microprocessor (14) are sent to a display unit (16′) to allow the operator to evaluate his/her physical conditions. The display unit (16′) may be a liquid crystal display. The control unit (24′) is mounted on the tube (42) and the power module (18′) is mounted inside the tube (42).

It is noted that the physiological parameter detection/display assembly of the present invention is compact in dimension and simple in compositions. Furthermore, the physiological parameters from the calculation of the microprocessor provide instant evaluation of body conditions so that the operator is able to monitor his/her physical conditions and take precautions if any of the parameters is not normal.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A portable physiological parameter detection/display assembly comprising:
an optical sensor for engagement with an operator to acquire a pulse signal;
an A/D converter converting the pulse signal into a digital format;
a microprocessor for receiving the digital format pulse signal and proceeding with calculation of at least one heart rate variability parameter;
a display to display the parameter; and
a power module to provide necessary electricity to the assembly.

2. The assembly as claimed in claim 1, wherein the optical sensor has at least one light emitter for projecting light to the operator's tissue and at least one light detector for receiving reflection light from the operator's tissue.

3. The assembly as claimed in claim 1 further comprising a storage unit is electrically connected to the microprocessor to store the pulse signal and the heart rate variability parameter.

4. The assembly as claimed in claim 2 further comprising a storage unit is electrically connected to the microprocessor to store the pulse signal and the heart rate variability parameter.

5. The assembly as claimed in claim 1, wherein the power module is a battery.

6. The assembly as claimed in claim 3, wherein the power module is a battery.

7. The assembly as claimed in claim 4, wherein the power module is a battery.

8. The assembly as claimed in claim 1 further comprising a timing unit connected to the microprocessor for providing time information.

9. The assembly as claimed in claim 3 further comprising a timing unit connected to the microprocessor for providing time information.

10. The assembly as claimed in claim 1 further comprising a control unit connected to the microprocessor.

11. The assembly as claimed in claim 3 further comprising a control unit connected to the microprocessor.

12. The assembly as claimed in claim 8 further comprising a control unit connected to the microprocessor.

13. The assembly as claimed in claim 8, wherein the power module is a battery.

14. The assembly as claimed in claim 10, wherein the power module is a battery.
